(19) United States
(12) Patent Application Publication
(10) Pub. No.: US 2005/0020779 A1
(43) Pub. Date: Jan. 27, 2005

(54) Block ethylenic copolymers cosmetic compositions containing them and cosmetic use of these copolymers

(76) Inventors: Nathalie Mougin, Paris (FR); Marco Vicic, Bry/Marne (FR); Colette Cazeneuve, Paris (FR); Bertrand Lion, Luzarches (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/495,424

(22) PCT Filed: Nov. 27, 2002
(86) PCT No.: PCT/FR02/04068

(30) Foreign Application Priority Data
Nov. 29, 2001 (FR) 01/15438

(51) Int. Cl. A61K 6/00; A61K 7/00

(52) U.S. Cl. 525/271; 424/401

(57) ABSTRACT

Linear block ethylenic copolymer comprising: at least two blocks with different glass transition temperatures (Tg); at least one of these blocks having a glass transition temperature of greater than or equal to 20° C.; said polymer also having mechanical parameters that satisfy at least one of the following three conditions: a Young's modulus E such that:
500 MPa≦E≦2000 MPa;
an ultimate strain εᵣ such that:
   5%≦εᵣ≦50%;
an ultimate strain energy wᵣ such that: 0.1×10⁵ J/m³<wᵣ<150×10⁵ J/m³. Cosmetic composition comprising said polymer. The copolymer makes it possible to improve the styling power and the hold of a hair lacquer, to increase the adhesion and the wear strength of a nail varnish and to improve the staying power of a makeup composition without the compositions being tacky.

### DESCRIPTION

The present invention relates to novel polymers of specific structure of block ethylenic copolymer type.

The present invention also relates to a composition, especially a cosmetic or pharmaceutical composition, in particular a hair composition, comprising said polymer of specific structure.

The invention also relates to the use of these polymers in cosmetics for treating the skin, the nails or the hair.

Many compositions, and in particular hair compositions known as hairstyling compositions, which are in the form of sprays, gels or mousses, contain resins or polymers.

These are, in particular, acrylic polymers with high glass transition temperatures (Tg), such as those described in document FR-A-2 439 798.

Such polymers afford, especially in hairstyling, hold of the hairstyle, but they have the drawback of having excessive friability, which does not allow good hold of the hairstyle over time.

In the case of varnishes, the polymers are not resistant to impacts.

To solve the problems posed by these polymers, plasticizers are also used in cosmetic compositions in order to lower the glass transition temperature. However, polymers tend to have “tacky” effects or, in the case of hairstyling, a reduction in the “styling”.

There is thus a need for a polymer, which, when it is included in a composition, in particular a cosmetic composition, is such that this composition does not have the drawbacks, defects, limitations and disadvantages of the compositions of the prior art.

There is in particular a need for a polymer and a composition containing it that has an optimum combination of rigidity and “tack” properties.

Thus, a hair composition comprising the polymer must be able to afford more hold while at the same time maintaining a natural effect.

In the case of a treatment of the nails, comprising the application of a glossy protective film, with the aim of withstanding mechanical attack, such a protective film, which contains the polymer, must be capable of outstanding mechanical abrasion resistance.

In the case of a skin treatment, the makeup, when used, and which includes the polymer, must adhere to the skin without making it taut and at the same time being comfortable.

In all cases and irrespective of the composition in which the polymer used is present, it is necessary for this polymer to give a product that does not feel tacky.

The aim of the present invention is to provide a polymer that satisfies, inter alia, the needs, criteria and requirements mentioned above and that solves the problems of the polymers of the prior art.

This aim and others are achieved, in accordance with the present invention, by a linear block ethylenic copolymer comprising:
at least two blocks with different glass transition temperatures (Tg);
at least one of these blocks having a glass transition temperature (Tg) of greater than or equal to 20° C.;
said copolymer also having mechanical parameters that satisfy at least one of the following three conditions:
a Young's modulus E such that:
   500 MPa≦E<2000 MPa;
an ultimate strain εᵣ such that:
   5%≦εᵣ≦50%;
an ultimate strain energy wᵣ such that: 0.1×10⁵ J/m³<wᵣ<150×10⁵ J/m³.

Advantageously, the copolymer according to the invention has mechanical parameters that satisfy at least two of the conditions mentioned above. Thus, the copolymer-according to the invention has, for example:
a Young's modulus E such that:
   500 MPa≦E≦2000 MPa;
an ultimate strain εᵣ such that:
   5%≦εᵣ≦50%.

Alternatively, the copolymer according to the invention has:
a Young's modulus E such that:
   500 MPa≦E≦2000 MPa; and
an ultimate strain energy wᵣ such that: 0.1×10⁵ J/m³<wᵣ<150×10⁵ J/m³.

Alternatively, the copolymer according to the invention has:
an ultimate strain εᵣ such that:
   5%≦εᵣ≦50%; and
an ultimate strain energy wᵣ such that: 0.1×10⁵ μm³<wᵣ<150×10⁵ μm³.

Preferably, the copolymer according to the invention satisfies all three of the conditions mentioned, as regards its mechanical parameters, indicated above.

A subject of the invention is also cosmetic compositions comprising said linear block ethylenic copolymers.

When they are incorporated into such compositions, the copolymers having the specific structure according to the invention afford extremely advantageous properties, or rather a combination of extremely advantageous properties, which it was not possible to obtain with the polymers of the prior art.

In general, the polymers have an optimum combination of rigidity and of tack-free nature and they thus lead to compositions or systems especially having improved mechanical strength, wear resistance and hold over time, and reduced fragility, while at the same time not being tacky.

Thus, when the copolymers according to the invention are used in compositions for treating the hair, such as lacquers, they afford greater hold over time. They are less fragile than a conventional lacquer and they are at the same time not tacky.

In nail varnishes, the varnish comprising the copolymer according to the invention has greater wear resistance and is not tacky.

In makeup products, for instance lipsticks or foundations, the makeup shows good staying power on the lips or the skin, without giving a tacky feel.

The invention also relates to a cosmetic makeup or care process for keratin materials, comprising the application to the keratin materials of a composition according to the invention.

The invention thus also relates to the use of the copolymers according to the invention to improve the styling power and the hold of a hair lacquer, the use of the copolymers to improve the adhesion and the wear resistance of a nail varnish and, finally, the use of the copolymers to increase the adhesion of a makeup composition.

The copolymers according to the invention thus provide a solution to the problems posed by the polymers of the prior art.

The unexpected advantageous properties of the specific copolymers of the invention, which are fundamentally linear copolymers, arise especially from the specific nature of the blocks from which they are formed, which are defined by particular glass transition temperatures.

There was nothing in the prior art to suggest that by using a specifically linear copolymer, by setting defined Tg conditions for the blocks from which the copolymer is formed, and by establishing specific ranges for at least one mechanical parameter defining this copolymer, it would be possible, according to the invention, to obtain a combination of excellent properties for this copolymer.

Without wishing to be bound by any theory, the advantageous properties of the copolymer according to the invention are thought to arise from the fact that, firstly, it is linear, and that, secondly, the nature of the blocks is specifically chosen so as to promote phase separation between the blocks and thus, inter alia, to afford optimum control of the rigidity and tack of the copolymer.

More specifically, the copolymers according to the invention are block copolymers. This term generally means that the copolymers consist of sequences or blocks covalently attached together.

In addition, two successive blocks are of different nature. However, two non-successive blocks may be of the same nature. Each block may consist of a homopolymer or a copolymer, this homopolymer or copolymer in turn possibly being random or alternating.

The copolymers of the invention are defined as being ethylenic copolymers. This means that the monomers from which the sequences or blocks from which the copolymer is formed are monomers containing a carbon-carbon unsaturated double bond of ethylenic type.

In addition, specifically, the copolymer according to the invention is a linear copolymer. This means that the invention is not intended to cover copolymers with a non-linear structure, for example a branched, starburst, grafted or other structure. The linear nature of the copolymers of the invention is important for giving the compositions containing it the advantageous properties described above.

Advantageously, the copolymer is a film-forming polymer, i.e. it is capable, by itself or in the presence of an auxiliary film-forming agent, at a temperature ranging from 20° C. to 30° C., of forming a continuous film (viewed by the naked eye) that adheres to a keratin support.

According to the invention, the copolymer comprises at least two sequences or blocks that have different glass transition temperatures (Tg) and, also, at least one of these sequences or blocks of the copolymer has a glass transition temperature of greater than or equal to 20° C.

Since the glass transition temperature Tg is an essential parameter for defining the blocks of the copolymer of the invention and, consequently, the copolymer of the invention, it is important to indicate that the glass transition temperatures of the blocks of the copolymers used in the present invention are measured by differential thermal analysis (DSC, “Differential Scanning Calorimetry”) for the dry polymer, at a heating rate of 10° C./minute.

The polymer according to the invention is also defined by mechanical parameters: Young's modulus, or rigidity E, ultimate strain εᵣ, ultimate strain energy wᵣ, at least one of which is in a specific range.

It is thus fundamental to define the methods according to which these three mechanical parameters are determined.

First, it should be pointed out that these parameters relate to a film obtained by drying a solution of the copolymer in a solvent that is suitable for said copolymer, for example ethanol, at room temperature and at a relative humidity of 50%.

For the purposes of the present invention, the expression “film obtained by drying at room temperature and at a relative humidity of 50%±5%” means a film obtained by drying at 22±2° C. after a drying time of two days, the amount of solution being adapted to obtain in a Teflon matrix a film 250±50 μm thick.

For the purposes of the present invention, the Young's modulus E, the ultimate strain (εᵣ) and the ultimate strain energy (wᵣ) are defined by means of the tests described below. To perform the tensile tests, the film is cut into dumbbell-shaped specimens with a working length of 33±1 mm and a working width of 6 mm. The cross section (S) of the specimen is then defined as: S=width×thickness (mm²); this cross section (S) will be used to calculate the stress.

The tests are performed on a tensile testing machine equipped with an optical extensometer for measuring the draw, sold under the name Lloyd® LR5K or sold under the name Zwick® Z010. The measurements are performed under the same temperature and humidity conditions as for drying, i.e. a temperature of 22±2° C. and a relative humidity of 50±5%.

The specimens are drawn at a draw rate of 2 mm/minute.

A draw speed is thus applied and the length (L) of the specimen and the force F required to impose this length are simultaneously measured. From these data L and F, the stress σ and strain parameters are determined.

The distance (L) is measured with an optical extensometer using adhesive pellets placed on the dumbbell specimen. The initial distance between these two pellets defines the working length Lo used to calculate the strain ε.

A curve of the stress σ(=F/S) as a function of the strain ε(=(L/Lo)*100) is thus obtained, the test being performed until the specimen fails.

The ultimate strain εᵣ is the maximum strain of the sample before the point of failure (in %).

The ultimate strain energy wᵣ in J/m³ is defined as being the area under this stress/strain curve such that:

$$W = ( {\int }_{0}^{{L}_{max}} F ⅆ L ) / ( Lo \times S ) ,$$

in which formula Lo is in metres and S is in m².

Finally, E corresponds to the slope of the curve σ=f(ε) in the linear portion of the curve.

Besides the conditions relating to the Tg of their blocks, the copolymers of the invention are therefore defined by a Young's modulus or rigidity E, which is given in MPa and which corresponds to the slope of the curve σ=f(ε), considered in the linear portion of this curve (start of the test).

According to the invention, E satisfies the relationship 500 MPa≦E≦2000 MPa, preferably 600 MPa≦E≦2000 MPa and more preferably 800 MPa≦E≦2000 MPa.

Alternatively, the copolymers of the invention are defined by an ultimate strain εᵣ, which is given in % and which corresponds to the maximum strain of the copolymer sample before the point of failure.

According to the invention, εᵣ satisfies the relationship 5%≦εᵣ≦50%, preferably 8%≦εᵣ≦50%, and more preferably 10%≦εᵣ≦50%.

Alternatively, the copolymers of the invention are defined by an ultimate strain energy wᵣ, which is given in J/m³ and which corresponds to the total energy absorbed per unit volume of the sample up to the point of failure.

According to the invention, wᵣ satisfies the relationship 0.1×10⁵ J/m³<wᵣ<150×10⁵ μm³, preferably 0.5×10⁵ J/m³<wᵣ<150×10⁵ J/m³ and more preferably 1×10⁵⁹ J/m³<wᵣ<150×10⁵ J/m³.

The copolymers according to the invention may also be defined as being copolymers such that at least two of the mechanical parameters defined above are within the ranges indicated above.

The copolymers according to the invention may, finally, be copolymers for which the three mechanical parameters listed above satisfy all of the mentioned relationships.

Each block of the copolymer according to the invention is derived from one type of monomer or from several different types of monomer.

This means that each block may consist of a homopolymer or a copolymer; this copolymer constituting the block may in turn be random or alternating.

According to the invention, the copolymer comprises at least two blocks with different glass transition temperatures (Tg). Advantageously, the difference in glass transition temperature between these two blocks with different glass transition temperatures is generally from 40 to 120° C., preferably from 40 to 110° C. and more preferably from 40 to 100° C.

The number-average mass of the copolymer is generally from 10 000 to 500 000 and preferably from 50 000 to 200 000.

Advantageously, the proportion of the block with a Tg of greater than or equal to 20° C. is from 99% to 40% of the polymer, preferably from 95% to 55% and more preferably from 90% to 50%.

Advantageously, the block with a Tg of greater than or equal to 20° C. has a Tg temperature of from 20 to 200° C., preferably from 20 to 170° C. and more preferably from 20 to 150° C.

The block whose glass transition temperature is greater than or equal to 20° C., which is a homopolymer or a copolymer, is preferably totally or partly derived from one or more monomers, which is (are) such that the homopolymers prepared from these monomers have glass transition temperatures of greater than or equal to 20° C.

More preferably, the block whose glass transition temperature is greater than or equal to 20° C. is a homopolymer, consisting of only one type of monomer (for which the Tg of the corresponding homopolymer is greater than 20° C.).

The monomers whose homopolymers have glass transition temperatures of greater than or equal to 20° C. and from which is (are) preferably derived the block(s) with a Tg≧20° C. of the copolymer of the invention are preferably chosen from the following monomers:
the vinyl compounds of formula:
   CH₂═CH—R₁,
in which R₁ is a hydroxyl group; a group 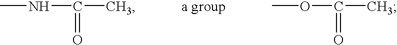
   a C₃ to C₈ cycloalkyl group; a C₆ to C₂₀ aryl group; a C₇-C₃₀ aralkyl group (C₁ to C₄ alkyl group); a 4- to 12-membered heterocyclic group containing one or more hetero atoms chosen from O, N and S; a heterocyclylalkyl group. (C₁ to C₄ alkyl) such as a furfuryl group; said cycloalkyl, aryl, aralkyl, heterocyclic or heterocyclylalkyl groups possibly being optionally substituted with one or more substituents chosen from hydroxyl groups, halogen atoms and linear or branched 1 to 4 C alkyl groups in which is (are) optionally interpolated one or more hetero atoms chosen from O, N, S and P, and said alkyl groups also possibly being optionally substituted with one or more substituents chosen from hydroxyl groups and halogen atoms (Cl, Br, I and F).

Examples of vinyl monomers include vinylcyclohexane, styrene and vinyl acetate.

The acrylates of formula CH₂═CH—COOR₂
in which R₂ is a tert-butyl group; a C₃ to C₈ cycloalkyl group; a C₆-C₂₀ aryl group; a C₇-C₃₀ aralkyl group (C₁ to C₄ alkyl group); a 4- to 12-membered heterocyclic group containing one or more hetero atoms chosen from O, N and S; a heterocyclylalkyl group (C₁ to C₄ alkyl) such as a furfuryl group; said cycloalkyl, aryl, aralkyl, heterocyclic or heterocyclylalkyl groups possibly being optionally substituted with one or, more substituents chosen from hydroxyl groups, halogen atoms and linear or branched alkyl groups of 1 to 4 C in which is (are) optionally interpolated one or more hetero atoms chosen from O, N, S and P, said alkyl groups also possibly being optionally substituted with one or more substituents chosen from hydroxyl groups and halogen atoms (Cl, Br, I and F).

Examples of acrylate monomers include tert-butylcyclohexyl, tert-butyl, tert-butylbenzyl, furfuryl and isobornyl acrylate;
the methacrylates of formula CH₂═C(CH₃)—COOR₃,
in which R₃ is a linear or branched alkyl group of 1 to 4 C, such as a methyl, ethyl, propyl or isobutyl group, said alkyl group also possibly being optionally substituted with one or more substituents chosen from hydroxyl groups and halogen atoms (Cl, Br, I and F); a C₃ to C₈ cycloalkyl group; a C₆-C₂₀ aryl group; a C₇-C₃₀ aralkyl group (C₁ to C₄ alkyl group); a 4- to 12-membered heterocyclic group containing one or more hetero atoms chosen from O, N and S; a heterocyclylalkyl group (alkyl of 1 to 4 C), such as a furfuryl group; said cycloalkyl, aryl, aralkyl, heterocyclic or heterocyclylalkyl groups possibly being optionally substituted with one or more substituents chosen from hydroxyl groups, halogen atoms and linear or branched alkyl groups of 1 to 4 C in which is (are) optionally interpolated one or more hetero atoms chosen from O, N, S and P, said alkyl groups also possibly being optionally substituted with one or more substituents chosen from hydroxyl groups and halogen atoms (Cl, Br, I and F).

Examples of methacrylate monomers include methyl, ethyl, n-butyl, isobutyl, t-butylcyclohexyl, t-butylbenzyl and isobornyl methacrylate;
the (meth)acrylamides of formula: 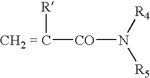
   in which R′ denotes H or —CH₃, and in which R₄- and R₅, which may be identical or different, each represent a hydrogen atom or a linear or branched alkyl group of 1 to 12 carbon atoms, such as an n-butyl, t-butyl, isopropyl, isohexyl, isooctyl or isononyl group.

Examples of (meth)acrylamide monomers include N-butylacrylamide, N-t-butylacrylamide, N-isopropylacrylamide, N,N-dimethylacrylamide and N,N-dibutylacrylamide.

The preferred monomers among all those mentioned above are chosen from furfuryl, isobornyl, tert-butyl, t-butylcyclohexyl and t-butylbenzyl acrylate, methyl, n-butyl, ethyl and isobutyl methacrylate, styrene, vinylacetate and vinylcyclo-hexane.

The block with a glass transition temperature greater than or equal to 20° C. may, besides the monomers indicated above, referred to as the “main monomers”, for which the glass transition temperature Tg of the corresponding homopolymer is greater than or equal to 20° C., comprises one or more other different monomers known as additional monomers, which, to be distinguished from the “main” monomers, have a Tg of the corresponding homopolymer of less than 20° C.

This or these additional monomer(s) is (are), of course, chosen such that the Tg of the block is greater than or equal to 20° C.

Thus, a block with an adequate Tg, of greater than or equal to 20° C., may be formed from a copolymer, this polymer consisting of a first monomer or main monomer for which the Tg of the corresponding homopolymer is in the range from 20° C. to 200° C., and of a second monomer or additional monomer for which the Tg of the corresponding homopolymer is in the range from below 20° C. to −100° C.

For example, a “main” monomer with a Tg (of the corresponding homopolymer) equal to 100° C. may be combined, in a proportion of 80% by weight, in the copolymer forming the block, with a monomer whose Tg is equal to −70° C., in a proportion of 20% by weight, and the resulting block will have a Tg by weight of 50° C.

These additional monomers which thus have a Tg of the equivalent homopolymer that is strictly below 20° C. are chosen from acrylates, methacrylates, (meth)acrylamide and vinyl and allylic compounds. Thus, the additional monomers may be chosen from the following monomers:
ethylenic hydrocarbons of 2 to 10 C, such as ethylene, isoprene and butadiene;
the acrylates of formula CH₂═CHCOOR₆, R₆ representing a linear or branched alkyl group of 1 to 12 C, with the exception of the tert-butyl group, in which is (are) optionally interpolated one or more hetero atoms chosen from O, N and S, said alkyl group also possibly being optionally substituted with one or more substituents chosen from hydroxyl groups and halogen atoms (Cl, Br, I and F),
examples of groups R₆ are methyl, ethyl, propyl, butyl, isobutyl, hexyl, ethylhexyl, octyl, lauryl, isooctyl, isodecyl, hydroxyethyl, hydroxypropyl, methoxyethyl, ethoxyethyl and methoxypropyl groups,
R₆ may also be a C₁ to C₁₂ alkyl-POE (polyoxy-ethylene) with repetition of the oxyethylene unit from 5 to 30 times, for example methoxy-POE,
R₆ may also be a polyoxyethylene group comprising from 5 to 30 ethylene oxide units;
the methacrylates of formula: 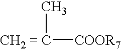
R₇ representing a linear or branched alkyl group of 3 to 12 C, in which is (are) optionally interpolated one or more hetero atoms chosen from O, N and S, said alkyl group also possibly being optionally substituted with one or more substituents chosen from hydroxyl groups and halogen atoms (Cl, Br, I or F); examples of groups R₂ are hexyl, ethylhexyl, octyl, lauryl, isooctyl, isodecyl, dodecyl, methoxyethyl, methoxypropyl, ethoxyethyl, POE (polyoxyethylene with repetition of the oxyethylene unit from 5 to 30 times) and (C₁ to C₃₀)alkyl-POE (with repetition of the oxyethylene unit from 5 to 30 times);
the vinyl esters of formula:
   R₈—CO—O—CH═CH₂
in which R₈ represents a linear or branched alkyl group of 2 to 12 C;
examples of such vinyl esters are: vinyl-propionate, vinylbutyrate, vinyl ethylhexanoate, vinyl neononanoate and vinyl neododecanoate;
ethers of vinyl and of alkyl of 1 to 12 C, such as methyl vinyl ether and ethyl vinyl ether;
N-(1 to 12 C)alkylacrylamides, such as N-octylacrylamide.

The monomers that are particularly preferred are: n-butyl acrylate, ethylhexyl acrylate, isobutyl acrylate, methoxyethyl acrylate and ethoxyethyl (meth)acrylate.

This or these additional monomers is (are) generally present in an amount of less than or equal to 50% by weight, preferably less than or equal to 45% by weight and more preferably less than or equal to 40% by weight relative to the total weight of the block with a Tg of greater than or equal to 20° C.

Advantageously, the copolymer according to the invention comprises at least one hydrophilic block comprising hydrophilic monomers.

The hydrophilic block may be defined as being a water-soluble or water-dispersible block.

The polymer forming the block is water-soluble if it is soluble in water to a proportion of at least 5% by weight at 25° C.

The polymer forming the block is water-dispersible if it forms, at a concentration of from 5% to 25%, a stable suspension of generally spherical fine particles. The mean size of the particles constituting said dispersion is less than 1 μm and more generally ranges between 5 and 400 nm and preferably from 10 to 250 nm. These particle sizes are measured by light scattering.

The hydrophilic block is preferably the block whose glass transition temperature is greater than or equal to 20° C., but it may also be a block whose glass transition temperature is less than 20° C.

It is known that hydrophilic monomers whose homopolymers have a glass transition temperature of less than 20° C. are not common.

Accordingly, the hydrophilic block, in the case where it is a block with a Tg of less than 20° C., is necessarily a copolymer.

This hydrophilic block therefore comprises one or more hydrophilic monomer(s) whose corresponding homopolymers have glass transition temperatures of greater than or equal to 20° C. and one or more other non-hydrophilic monomer(s) chosen especially from those whose homopolymers have Tg values of less than 20° C.

The proportion of the various hydrophilic and non-hydrophilic monomers is preferably chosen such that the block as a whole consisting of a copolymer has a Tg of less than 20° C. When the hydrophilic block has a glass transition temperature of greater than or equal to 20° C., it generally also comprises from 70% to 100% and preferably from 80% to 100% of hydrophilic monomers for which the Tg values of the corresponding homopolymers are greater than or equal to 20° C.

When the hydrophilic block has a glass transition temperature of less than 20° C., it generally comprises from 10% to 70% and preferably from 20% to 65% of hydrophilic monomers for which the Tg values of the corresponding homopolymers are greater than or equal to 20° C.

In the preferred case in which the hydrophilic block is the block with a Tg of greater than or equal to 20° C., this block will consist of a majority proportion of hydrophilic monomers which thus have a Tg value for the corresponding homopolymer of greater than or equal to 20° C., and a minority proportion of monomers for which the Tg of the corresponding homopolymer is less than 20° C.

More preferably, the hydrophilic block with a Tg of greater than or equal to 20° C. is a homopolymer consisting exclusively of hydrophilic monomers with a Tg of greater than or equal to 20° C.

Examples of hydrophilic monomers for which the Tg of the corresponding homopolymer is greater than 20° C. include cationic monomers, anionic monomers and nonionic monomers.

Examples of cationic monomers are:

-2-vinylpyridine;
-4-vinylpyridine;
dimethylaminoethyl methacrylate (DMAEMA);
diethylaminoethyl methacrylate (DEAEMA);
dimethylaminopropylacrylamide; and
salts thereof, whether they are salts of mineral acids, such as sulphuric acid or hydrochloric acid, or salts of an organic acid.

These organic acids may comprise one or more carboxylic, sulphonic or phosphonic groups. They may be linear, branched or cyclic aliphatic acids or alternatively aromatic acids. These acids may also comprise one or more hetero atoms chosen from O and N, for example in the form of hydroxyl groups.

An example of an acid containing an alkyl group is acetic acid CH₃COOH.

An example of a polyacid is terephthalic acid.

Examples of hydroxy acids are citric acid and tartaric acid.

Examples of anionic monomers are:
acrylic acid, methacrylic acid, crotonic acid, maleic anhydride, itaconic acid, fumaric acid and maleic acid;
styrenesulphonic acid, acrylamidopropane-sulphonic acid, vinylbenzoic acid or vinylphosphoric acid, and salts thereof.

The neutralizer may be a mineral base, such as LiOH, NaOH, KOH, Ca(OH)₂, or NH₄OH, an organic base, for example a primary, secondary or tertiary amine, such as a primary alkylamine, for instance amino-2-methyl-2-propanol, or a secondary or tertiary alkylamine.

Examples of nonionic monomers are:
hydroxyalkyl (meth)acrylates in which the alkyl group contains from 2 to 4 carbon atoms, in particular hydroxyethyl (meth)acrylate;
vinyllactams; (meth)acrylamides, N-(C₁-C₄)alkyl(meth)acrylamides, for instance isobutyl-acrylamide; and polysaccharide (meth)acrylates, for instance sucrose acrylate.

It should be noted that even if the copolymer comprises a hydrophilic block, the overall copolymer is not necessarily hydrophilic.

The copolymers may comprise two blocks with a Tg≧20° C. and one or two blocks with a Tg<20° C.

The linear block ethylenic copolymers according to the invention are chosen from:
diblock copolymers;
triblock copolymers;
multiblock copolymers containing more than three blocks.

In the case of multiblock copolymers, in which at least one block satisfies the criterion of Tg greater than or equal to 20° C., the other blocks or sequences then have a Tg of less than 20° C. and greater than or equal to −100° C.

The copolymers according to the invention may be prepared by anionic polymerization.

Preferably, however, the copolymers according to the invention are obtained, in a first mode, by controlled free-radical polymerization, but they may also be obtained, according to a second mode, by conventional free-radical polymerization.

First Mode:

The block copolymers according to the invention are preferably obtained by controlled free-radical polymerization, described especially in “New Method of Polymer Synthesis”, Blackie Academic & Professional, London, 1995, volume 2, page 1.

Controlled free-radical polymerization makes it possible to reduce the reactions that deactivate the growing free-radical species, in particular the termination step, these being reactions which, in standard free-radical polymerization, interrupt the growth of the polymer chain irreversibly and without control.

In order to reduce the probability of the termination reactions, it has been proposed to transiently and reversibly block the growing free-radical species, by forming “dormant” active species in the form of a bond with a low dissociation energy.

Thus, the polymerization may be performed according to the atom-transfer technique, or by reaction with a nitroxide, or alternatively according to the “reversible addition-fragmentation chain transfer” technique.

The technique of atom-transfer radical polymerization, also known under the abbreviation ATRP, consists in blocking the growing free-radical species in the form of a bond of C-halide type (in the presence of a metal/ligand complex). This type of polymerization is reflected by control of the mass of the polymers formed and by a low polydispersity index.

In general, atom-transfer radical polymerization is performed by polymerization of one or more free-radical-polymerizable monomers, in the presence of:
an initiator containing at least one transferable halogen atom;
a compound comprising a transition metal capable of participating in a reduction step with the initiator and a “dormant” polymer chain; and
a ligand which may be chosen from compounds comprising a nitrogen (N), oxygen (O), phosphorus (P) or sulphur (S) atom, which can become co-ordinated via a σ bond to said compound comprising a transition metal, the formation of direct bonds between said compound comprising a transition metal and the polymer in formation being avoided.

The halogen atom is preferably a chlorine or bromine atom.

This process is described in particular in patent application WO 97/18247 and in the article by Matyjasezwski et al. published in JACS, 117, page 5614 (1995).

The technique of free-radical polymerization by reaction with a nitroxide consists in blocking the growing free-radical species in the form of a bond of C—ONR₁R₂ type, R₁ and R₂ possibly being, independently of each other, an alkyl radical containing from 2 to 30 carbon atoms or together forming, with the nitrogen atom, a ring containing from 4 to 20 carbon atoms, for instance a 2,2,6,6-tetramethylpiperidyl ring. This polymerization technique is described especially in the articles “Synthesis of nitroxy-functionalized polybutadiene by anionic polymerization using a nitroxy-functionalized terminator”, published in Macromolecules 1997, volume 30, pages 4238-4242, and “Macromolecular engineering via living free radical polymerizations” published in Macromol. Chem. Phys. 1998, vol. 199, pages 923-935, or alternatively in patent application WO-A-99/03894.

The technique of RAFT (reversible addition-fragmentation chain transfer) polymerization consists in blocking the growing free-radical species in the form of a bond of C—S type. Dithio compounds are used to do this, for instance thiobenzoates, dithio-carbamates or xanthan disulphides. This technique is described especially in patent application WO-A-98/58974 and in the article “A more versatile route to block copolymers and other polymers of complex architecture by living radical polymerization: the RAFT process”, published in Macromolecules, 1999, volume 32, pages 2071-2074.

Second Mode:

The block polymers according to the invention may also be obtained by using the standard free-radical polymerization technique, by sequentially casting the monomers. In this case, only the control of the nature of the blocks is possible (no control of the masses).

This is a matter of polymerizing, in a first stage, a monomer M1 in a polymerization reactor, monitoring, kinetically, its consumption over time and then, when M1 has been about 95% consumed, introducing a new monomer M2 into the polymerization reactor.

A polymer of block structure of M1-M2 type is thus readily obtained.

The invention also relates to cosmetic or pharmaceutical compositions comprising the copolymer of specific structure as has been described above.

Generally, these compositions contain from 0.1% to 60% by weight, preferably from 0.5% to 50% by weight and more preferably from 1% to 40% by weight of the copolymer according to the invention.

These cosmetic compositions according to the invention comprise, besides said polymers, a physiologically acceptable medium, i.e. a medium that is compatible with keratin materials, for instance the skin, the hair, the eyelashes, the eyebrows and the nails.

In general, it should be considered that the whole composition is physiologically acceptable.

Said physiologically acceptable medium generally comprises a suitable physiologically acceptable solvent, in which the copolymer according to the invention is present in dissolved or dispersed form.

The composition may thus comprise, as solvent forming a hydrophilic phase, water or a mixture of water and of hydrophilic organic solvent(s), for instance alcohols and especially linear or branched lower monoalcohols containing from 2 to 5 carbon atoms, for instance ethanol, isopropanol or n-propanol, and polyols, for instance glycerol, diglycerol, propylene glycol, sorbitol, pentylene glycol and polyethylene glycols. The hydrophilic phase may further contain hydrophilic C₂ ethers and C₂-C₄ aldehydes. The water or the mixture of water and of hydrophilic organic solvents may be present in the composition according to the invention in a content ranging from 0% to 90% (especially 0.1% to 90%) by weight and preferably from 0% to 60% by weight (especially 0.1% to 60% by weight) relative to the total weight of the composition.

The composition may further comprise a fatty phase, consisting especially of fatty substances that are liquid at room temperature (in general 25° C.) and/or fatty substances that are solid at room temperature, such as waxes, pasty fatty substances and gums, and mixtures thereof. These fatty substances may be of animal, plant, mineral or synthetic origin. This fatty phase may also contain lipophilic organic solvents.

As fatty substances that are liquid at room temperature, often known as oils, which may be used in the invention, mention may be made of: hydrocarbon-based oils of animal origin such as perhydrosqualene; hydrocarbon-based plant oils such as liquid triglycerides of fatty acids containing from 4 to 10 carbon atoms, for instance heptanoic or octanoic acid triglyceride, or alternatively sunflower oil, corn oil, soybean oil, grape seed oil, sesame seed oil, apricot oil, macadamia oil, castor oil, avocado oil, caprylic/capric acid triglycerides, jojoba oil and shea butter; linear or branched hydrocarbons of mineral or synthetic origin such as liquid paraffins and derivatives thereof, petroleum jelly, polydecenes and hydrogenated polyisobutene such as parleam; synthetic esters and synthetic ethers, especially of fatty acids, such as, for example, purcellin oil, isopropyl myristate, 2-ethylhexyl palmitate, 2-octyldodecyl stearate, 2-octyldodecyl erucate and isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate or diethylene glycol diisononanoate; and pentaerythritol esters; fatty alcohols containing from 12 to 26 carbon atoms, for instance octyldodecanol, 2-butyloctanol, 2-hexyldecanol, 2-undecylpentadecanol and oleyl alcohol; partially hydrocarbon-based or silicone-based fluoro oils; silicone oils, for instance linear or cyclic, volatile or non-volatile polydimethylsiloxanes (PDMSs) that are liquid or pasty at room temperature, for instance cyclomethicones, dimethicones, optionally comprising a phenyl group, for instance phenyl trimethicones, phenyltrimethylsiloxydiphenyl siloxanes, diphenylmethyldimethyltrisiloxanes, diphenyl dimethicones, phenyl dimethicones and polymethylphenylsiloxanes; mixtures thereof.

These oils may be present in a content ranging from 0.01% to 90% and better still from 0.1% to 85% by weight, relative to the total weight of the composition.

The composition according to the invention may further comprise one or more organic solvents that are cosmetically acceptable (acceptable tolerability, toxicology and feel). These solvents may generally be present in a content ranging from 0% to 90%, preferably from 0.1% to 90% and more preferably from 10% to 90% by weight, and better still from 30% to 90%, relative to the total weight of the composition.

As solvents that may be used in the composition of the invention, mention may be made of acetic acid esters, for instance methyl acetate, ethyl acetate, butyl acetate, amyl acetate, 2-methoxyethyl acetate or isopropyl acetate; ketones, for instance methyl ethyl ketone or methyl isobutyl ketone; hydrocarbons, for instance toluene, xylene, hexane or heptane; aldehydes containing from 5 to 10 carbon atoms; ethers containing at least 3 carbon atoms; and mixtures thereof.

The waxes may be hydrocarbon-based waxes, fluoro waxes and/or silicone waxes and may be of plant, mineral, animal and/or synthetic origin. In particular, the waxes have a melting point of greater than 25° C. and preferably greater than 45° C.

As waxes that may be used in the composition of the invention, mention may be made of beeswax, carnauba wax or candelilla wax, paraffin, microcrystalline waxes, ceresin or ozokerite; synthetic waxes, for instance polyethylene waxes or Fischer-Tropsch waxes, or silicone waxes, for instance alkyl dimethicones or alkoxy dimethicones containing from 16 to 45 carbon atoms.

The gums are generally polydimethylsiloxanes (PDMSs) of high molecular weight or cellulose gums or polysaccharides and the pasty substances are generally hydrocarbon-based compounds, for instance lanolins and derivatives thereof, or PDMSs.

The nature and amount of the solid substances depend on the desired mechanical properties and textures. As a guide, the composition may contain from 0 to 50% by weight and better still from 1% to 30% by weight of waxes, relative to the total weight of the composition.

The polymer may be combined with one or more auxiliary film-forming agents. Such a film-forming agent may be chosen from any compound known to those skilled in the art as being capable of fulfilling the desired function, and may be chosen especially from plasticizers and coalescers.

The composition according to the invention may further comprise one or more dyestuffs chosen from water-soluble dyes and pulverulent dyestuffs, for instance pigments, nacres and flakes that are well known to those skilled in the art. The dyestuffs may be present in the composition in a content ranging from 0.01% to 50% by weight and preferably from 0.01% to 30% by weight, relative to the weight of the composition.

The term “pigments” should be understood as meaning white or coloured, mineral or organic particles of any form, which are insoluble in the physiological medium and which are intended to colour the composition.

The term “nacres” should be understood as meaning iridescent particles of any form, produced especially in the shell of certain molluscs, or alternatively synthesized.

The pigments may be white or coloured, and mineral and/or organic. Among the mineral pigments that may be mentioned are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders, for instance aluminium powder or copper powder.

Among the organic pigments that may be mentioned are carbon black, pigments of D & C type, and lakes based on cochineal carmine or on barium, strontium, calcium or aluminium.

The nacreous pigments may be chosen from white nacreous pigments such as titanium-coated mica or bismuth oxychloride-coated mica, coloured nacreous pigments such as titanium mica coated with iron oxides, titanium mica coated especially with ferric blue or with chromium oxide, titanium mica coated with an organic pigment of the abovementioned type and also nacreous pigments based on bismuth oxychloride.

The water-soluble dyes are, for example, beetroot juice or methylene blue.

The composition according to the invention may further comprise one or more fillers, especially in an amount ranging from 0.01% to 50% by weight and preferably ranging from 0.01% to 30% by weight, relative to the total weight of the composition. The term “fillers” should be understood as meaning colourless or white, mineral or synthetic particles of any form, which are insoluble in the medium of the composition, irrespective of the temperature at which the composition is manufactured. These fillers serve especially to modify the rheology or texture of the composition.

The fillers may be mineral or organic of any form, platelet, spherical or oblong, irrespective of the crystallographic form (for example lamellar, cubic, hexagonal, orthorhombic, etc.). Mention may be made of talc, mica, silica, kaolin, polyamide powder (Nylon®) (Orgasol® from Atochem), poly-β-alanine powder and polyethylene powder, tetrafluoroethylene polymer (Teflon®) powders, lauroyllysine, starch, boron nitride, hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (for example Tospearls® from Toshiba), polyorganosiloxane elastomer particles, precipitated calcium carbonate, magnesium carbonate, magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate.

The composition according to the invention may further contain ingredients commonly used in cosmetics, such as vitamins, thickeners, trace elements, softeners, sequestering agents, fragrances, acidifying or basifying agents, preserving agents, sunscreens, surfactants, antioxidants, agents for preventing hair loss, antidandruff agents and propellants, or mixtures thereof.

Needless to say, a person skilled in the art will take care to select this or these optional additional compound(s), and/or the amount thereof, such that the advantageous properties of the corresponding composition according to the invention are not, or are not substantially, adversely affected by the envisaged addition.

The composition according to the invention may especially be in the form of a suspension, a dispersion, a solution, a gel, an emulsion, especially an oil-in-water (O/W) or water-in-oil (W/O) emulsion, or a multiple emulsion (W/O/W or polyol/o/W or O/W/O emulsion), in the form of a cream, a paste, a mousse, a dispersion of vesicles, especially of ionic or nonionic lipids, a two-phase or multi-phase lotion, a spray, a powder, a paste, especially a soft paste (especially a paste having a dynamic viscosity at 25° C. of about from 0.1 to 40 Pa.s at a shear rate of 200 s⁻¹ after measurement for 10 minutes in cone/plate geometry). The composition may be anhydrous, for example it may be an anhydrous paste.

A person skilled in the art may select the appropriate presentation form, and also the method for preparing it, on the basis of his general knowledge, taking into account firstly the nature of the constituents used, especially their solubility in the support, and secondly the intended use of the composition.

The composition according to the invention may be a makeup composition, for instance complexion products (foundations), rouges, eye shadows, lip products, concealer products, blushers, mascaras, eyeliners, eyebrow makeup products, lip pencils, eye pencils, nail products, such as nail varnishes, body makeup products or hair makeup products (hair lacquer or mascara).

The composition according to the invention may also be a hair product, especially for holding the hairstyle or for shaping the hair. The hair compositions are preferably shampoos, hairsetting gels or lotions, blow-drying lotions, or fixing and styling compositions such as lacquers or sprays.

The lotions may be packaged in various forms, especially in vaporizers, in pump-dispenser bottles or in aerosol containers in order to allow the composition to be applied in vaporized form or in the form of a foam. Such packaging forms are indicated, for example, when it is desired to obtain a spray or a foam for fixing or treating the hair.

The invention will now be described with reference to the following examples, which are given as non-limiting illustrations.

### EXAMPLES

In Example 1 below, a polystyrene-b-polybutyl methacrylate copolymer is prepared via anionic polymerization.

Examples 2 and 3 are examples of cosmetic compositions including the copolymer prepared in Example 1.

### Example 1

Reagents Used

The monomers are purified by distillation over CaH₂ and stored under nitrogen.

The monomer M1 is styrene (M=104) onto the chains of which will be attached a 1,1-diphenylethylene unit (M=180) per chain, in order to give a living anion.

The monomer M2 is butyl methacrylate (M=142).

The solvent used is tetrahydrofuran (THF).

The initiator is sec-butyllithium (M=64).

Procedure

A flame-dried reactor, under nitrogen, equipped with a stirrer and a thermometer is used.

The solvent is transferred through a septum, using a syringe.

The initiator, dissolved in THF, is introduced dropwise at −30° C. until a red colour persists.

The distilled monomer M1 is added using a dropping funnel.

The polymerization takes place over 15 minutes at −78° C.

The living polystyrene anion then reacts with the 1,1-diphenylethylene for 15 minutes at −78° C., in order to attach a diphenylethylene unit per chain.

Next, the second monomer M2 is added. It polymerizes for one hour at −78° C., at the end of the living PS.

The reaction is quenched by adding methanol.

The polymer is precipitated from a 70/30 methanol/water mixture at −30° C.

The polymer is then dried for two days.

The following amounts of reagents are used:
initiator (sec-butyllithium): 1×10⁻² mol=0.64 g;
monomer 1 (styrene): 70×10⁻² mol=72.80 g;
1,1-diphenylethylene: 1×10⁻² mol=1.8 g;
monomer 2 (butyl methacrylate): 30×10-2 mol=42.6 g.

The copolymer that is obtained according to the invention comprises a polystyrene block with a Tg equal to 100° C., and a poly(butyl methacrylate) block with a Tg equal to 24° C.

Determination of the Masses and of the Ratio of the Blocks

The molecular mass is determined by gas chromatography (GC) in THF with a refractometric detector and standard columns, with a polystyrene standard.

The percentage of the two blocks is determined by ¹H NMR.

The number-average molecular mass Mn of the copolymer prepared above is 107 300; the ratio DPn=Mw/Mn=1.05 and the weight percentage of polystyrene relative to the total weight is 70%.

Characterization of the Mechanical Properties of the Copolymer Obtained

The copolymer prepared above is dissolved in methyl ethyl ketone to measure its mechanical properties: Young's modulus (E), ultimate strain (εᵣ) and ultimate strain energy (wᵣ), via the methods described in detail above.

The following results are obtained:
E=1 300 MPa;
εᵣ=2%;
wᵣ=4×10⁵ J/m³.

### Example 2

In this example, a “spray” is prepared by dissolving the polymer prepared in Example 1 in ethanol in a proportion of 6% by weight.

The composition is then applied to a hair.

Non-tacky styling with good hold over time is thus obtained. The composition applied is also less fragile than a standard lacquer containing a polymer not in accordance with the invention.

### Example 3

The polymer of Example 1 is dissolved in ethyl acetate, in a proportion of 25% by weight.

A varnish is thus obtained, the wear of which is reduced when compared with a standard solvent-based varnish containing a polymer not in accordance with the invention.

1. Linear block ethylenic copolymer comprising:
at least two blocks with different glass transition temperatures (Tg);
at least one of these blocks having a glass transition temperature of greater than or equal to 20° C.;
said polymer also having mechanical parameters that satisfy at least one of the following three conditions:
a Young's modulus E such that:
500 MPa≦E≦2000 MPa;
an ultimate strain εᵣ such that:
5%≦εᵣ≦50%;
an ultimate strain energy wᵣ such that: 0.1×10⁵ μm³<wᵣ<150×10⁵ J/m³.

2. Copolymer according to claim 1, the mechanical parameters of which satisfy at least two of the listed conditions.

3. Copolymer according to claim 2, which has:
a Young's modulus E such that:
500 MPa≦E≦2000 MPa; and
an ultimate strain εᵣ such that: 5%≦εᵣ≦50%.

4. Copolymer according to claim 2, which has:
a Young's modulus E such that:
500 MPa≦E≦2000 MPa; and
an ultimate strain energy wᵣ such that: 0.1×10⁵ J/m³<wᵣ<150×10⁵ J/m³.

5. Copolymer according to claim 2, which has:
an ultimate strain εᵣ such that: 5%≦εᵣ≦50%; and
an ultimate strain energy wᵣ such that: 0.1×10⁵ J/m³<wᵣ<150×10⁵ J/m³.

6. Copolymer according to claim 1, the mechanical parameters of which satisfy all three of the listed conditions.

7. Copolymer according to claims 1, which is a film-forming polymer.

8. Copolymer according to claims 1, in which the Young's modulus satisfies the relationship 600 MPa<E<2000 MPa.

9. Copolymer according to claims 1, in which εᵣ satisfies the relationship 8%≦εᵣ≦50%.

10. Copolymer according to claim 1, in which wᵣ satisfies the relationship 0.5×10⁵ J/m³<wᵣ<150×10⁵ J/m³.

11. Copolymer according to claim 1, in which the difference in glass transition temperatures (Tg) between the two blocks having different glass transition temperatures is from 40 to 120° C.

12. Copolymer according to claim 11, in which the difference in glass transition temperatures (Tg) between the two blocks having different glass transition temperatures is from 40 to 110° C.

13. Copolymer according to claim 12, in which the difference in glass transition temperatures (Tg) between the two blocks having different glass transition temperatures is from 40 to 100° C.

14. Copolymer according to claim 1, the number-average mass of which is from 10 000 to 500 000.

15. Copolymer according to claim 1, in which the proportion of the block with a glass transition temperature of greater than or equal to 20° C. is from 99% to 40% by mass of the copolymer.

16. Copolymer according to claim 15, in which the proportion of the block with a glass transition temperature of greater than or equal to 20° C. is from 95% to 55% by mass of the copolymer.

17. Copolymer according to claim 1, for which the block with a Tg of greater than or equal to 20° C. has a Tg from 20° C. to 200° C.

18. Copolymer according to claim 17, for which the block with a Tg of less than or equal to 20° C. has a Tg from 20° C. to 170° C.

19. Copolymer according to claim 1, for which the block with a Tg of greater than or equal to 20° C., which is a homopolymer or a copolymer, is totally or partly derived from one or more monomers, which is (are) such that the homopolymers prepared from these monomers have glass transition temperatures of greater than or equal to 20° C.

20. Copolymer according to claim 19, in which the block whose glass transition temperature is greater than or equal to 20° C. is a homopolymer consisting of a single type of monomer of Tg≧20° C.

21. Copolymer according to claim 19, which the monomers whose homopolymers have glass transition temperatures of greater than or equal to 20° C. are chosen from the following monomers:
the vinyl compounds of formula:
CH₂═CH—R₁,
in which R₁ is a hydroxyl group; a group 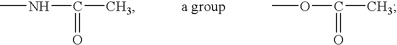
a C₃ to C₈ cycloalkyl group; a C₆ to C₂₀ aryl group; a C₇ to C₃₀ aralkyl group (C₁ to C₄ alkyl group); a 4- to 12-membered heterocyclic group containing one or more hetero atoms chosen from O, N and S; a heterocyclylalkyl group (C₁ to C₄ alkyl) such as a furfuryl group; said cycloalkyl, aryl, aralkyl, heterocyclic or heterocyclylalkyl groups possibly being optionally substituted with one or more substituents chosen from hydroxyl groups, halogen atoms and linear or branched 1 to 4 C alkyl groups in which is (are) optionally interpolated one or more hetero atoms chosen from O, N, S and P, and said alkyl groups also possibly being optionally substituted with one or more substituents chosen from hydroxyl groups and halogen atoms (Cl, Br, I and F);
the acrylates of formula CH₂═CH—COOR₂
in which R₂ is a tert-butyl group; a C₃ to C₈ cycloalkyl group; a C₆ to C₂₀ aryl group; a C₇ to C₃₀ aralkyl group (C₁ to C₄ alkyl group); a 4- to 12-membered heterocyclic group containing one or more hetero atoms chosen from O, N and S; a heterocyclylalkyl group (C₁ to C₄ alkyl) such as a furfuryl group; said cycloalkyl, aryl, aralkyl, heterocyclic or heterocyclylalkyl groups possibly being optionally substituted with one or more substituents chosen from hydroxyl groups, halogen atoms and linear or branched alkyl groups of 1 to 4 C in which is (are) optionally interpolated one or more hetero atoms chosen from O, N, S and P, said alkyl groups also possibly being optionally substituted with one or more substituents chosen from hydroxyl groups and halogen atoms (Cl, Br, I and F);
the methacrylates of formula CH₂═C(CH₃)—COOR₃,
in which R₃ is a linear or branched alkyl group of 1 to 4 C, such as a methyl, ethyl, propyl or isobutyl group, said alkyl group also possibly being optionally substituted with one or more substituents chosen from hydroxyl groups and halogen atoms (Cl, Br, I and F); a C₃ to C₈ cycloalkyl group; a C₆ to C₂₀ aryl group; a C₇ to C₃₀ aralkyl group (C₁ to C₄ alkyl group); a 4- to 12-membered heterocyclic group containing one or more hetero atoms chosen from O, N and S; a heterocyclylalkyl group (alkyl of 1 to 4 C), such as a furfuryl group; said cycloalkyl, aryl, aralkyl, heterocyclic or heterocyclylalkyl groups possibly being optionally substituted with one or more substituents chosen from hydroxyl groups, halogen atoms and linear or branched alkyl groups of 1 to 4 C in which is (are) optionally interpolated one or more hetero atoms chosen from O, N, S and P, said alkyl groups also possibly being optionally substituted with one or more substituents chosen from hydroxyl groups and halogen atoms (Cl, Br, I and F);
the (meth)acrylamides of formula: 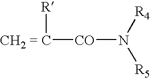
in which R′ denotes H or CH₃, and in which R₄ and R₅, which may be identical or different, each represent a hydrogen atom or a linear or branched alkyl group of 1 to 12 carbon atoms, such as an n-butyl, t-butyl, isopropyl, isohexyl, isooctyl or isononyl group.

22. Copolymer according to claim 21, in which the monomers whose homopolymers have glass transition temperatures of greater than or equal to 20° C. are chosen from furfuryl, isobornyl, tert-butyl, t-butylcyclohexyl and t-butylbenzyl acrylate, methyl, n-butyl, ethyl and isobutyl methacrylate, styrene, vinylacetate and vinylcyclohexane.

23. Copolymer according to claim 19, in which the block with a glass transition temperature of greater than or equal to 20° C. comprises, besides the monomer(s) for which the glass transition temperatures of the homopolymers prepared therefrom are greater than or equal to 20° C., one or more other different monomers or additional monomers for which the Tg values of the corresponding homopolymers are less than 20° C.

24. Copolymer according to claim 23, in which said additional monomer(s) is (are) chosen from:
ethylenic hydrocarbons of 2 to 10 C, such as ethylene, isoprene and butadiene;
the acrylates of formula CH₂═CHCOOR₆, R₆ representing a linear or branched alkyl group of 1 to 12 C, with the exception of the tert-butyl group, in which is (are) optionally interpolated one or more hetero atoms chosen from O, N and S, said alkyl group also possibly being optionally substituted with one or more substituents chosen from hydroxyl groups and halogen atoms (Cl, Br, I and F), or R₆ is a C₁ to C₁₋₂ alkyl-POE (polyoxyethylene) with repetition of the oxyethylene unit from 5 to 30 times, for example methoxy-POE, or R₆ is a polyoxyethylene group comprising from 5 to 30 ethylene oxide units;
the methacrylates of formula: 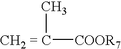
R₇ representing a linear or branched alkyl group of 3 to 12 C, in which is (are) optionally interpolated one or more hetero atoms chosen from O, N and S, said alkyl group also possibly being optionally substituted with one or more substituents chosen from hydroxyl groups and halogen atoms (Cl, Br, I or F);
the vinyl esters of formula:
R₈—CO—O—CH═CH₂
in which R₈ represents a linear or branched alkyl group of 2 to 12 C;
ethers of vinyl and of 1 to 12 C alkyl, such as methyl vinyl ether and ethyl vinyl ether;
N-(1 to 12 C)alkylacrylamides such as N-octylacrylamide.

25. Copolymer according to claim 23, in which said additional monomer(s) is(are) present in an amount of less than or equal to 50% by weight of the block with a Tg of greater than or equal to 20° C.

26. Copolymer according to claim 25, in which said additional monomer(s) is(are) present in an amount of less than or equal to 45% by weight of the block with a Tg of greater than or equal to 20° C.

27. Copolymer according to claim 23, in which the block with a Tg of greater than or equal to 20° C. is formed from a copolymer consisting of a first monomer for which the Tg of the corresponding homopolymer is in the range from 20° C. to 200° C. and of a second monomer or additional monomer for which the Tg of the corresponding homopolymer is in the range from less than 20° C. to −100° C.

28. Copolymer according to claim 1, comprising at least one hydrophilic block which comprises hydrophilic monomers.

29. Copolymer according to claim 28, in which said hydrophilic block is the block with a glass transition temperature of greater than or equal to 20° C.

30. Copolymer according to claim 28, in which the hydrophilic block is a block with a glass transition temperature of less than 20° C.

31. Copolymer according to claim 28, the hydrophilic block of which comprises one or more hydrophilic monomer(s) whose corresponding homopolymers have glass transition temperatures of greater than or equal to 20° C. and one or more other non-hydrophilic monomer(s) chosen especially from those whose homopolymers have Tg values of less than 20° C.

32. Copolymer according to claim 29, which the hydrophilic block comprises from 70% to 100% and preferably from 80% to 100% of hydrophilic monomers for which the Tg values of the corresponding homopolymers are greater than or equal to 20° C.

33. Copolymer according to claim 30, which the hydrophilic block comprises from 10% to 70% and preferably from 20% to 65% of hydrophilic monomers for which the Tg values of the corresponding homopolymers are greater than or equal to 20° C.

34. Copolymer according to claim 28, in which the hydrophilic monomers are chosen from cationic monomers, anionic monomers and nonionic monomers.

35. Copolymer according to claim 34, in which the cationic monomers are chosen from 2-vinylpyridine; 4-vinylpyridine; dimethylaminoethyl methacrylate (DMAEMA); diethylaminoethyl methacrylate (DEAEMA); dimethylaminopropylacrylamide; and salts thereof.

36. Copolymer according to claim 34, in which the anionic monomers are chosen from acrylic acid, methacrylic acid, crotonic acid, maleic anhydride, itaconic acid, fumaric acid, maleic acid, styrenesulphonic acid, acrylamidopropanesulphonic acid, vinylbenzoic acid and vinylphosphonic acid, and salts thereof.

37. Copolymer according to claim 34, in which the nonionic monomers are chosen from:
hydroxyalkyl (meth)acrylates in which the alkyl group contains from 2 to 4 carbon atoms, in particular hydroxyethyl (meth)acrylate;
vinyllactams;
(meth)acrylamides and N—(C₁ to C₄)alkyl(meth)acrylamides, for instance isobutylacrylamide;
polysaccharide (meth)acrylates, for instance sucrose acrylate.

38. Copolymer according to claim 1, chosen from diblock copolymers, triblock copolymers and multiblock copolymers containing more than three blocks.

39. Copolymer according to claim 38, which is a multiblock copolymer, in which two blocks have a Tg of greater than or equal to 20° C. and the other blocks have a Tg of less than 20° C. and greater than or equal to −100° C.

40. Cosmetic composition comprising the copolymer according to claim 1.

41. Cosmetic composition according to claim 40, containing from 0.1% to 60% by weight of the copolymer.

42. Composition according to claim 40, comprising, besides said copolymer, a physiologically acceptable medium in which the copolymer is in dissolved or dispersed form.

43. Composition according to claim 40, in which the physiologically acceptable medium comprises one or more suitable solvents forming a hydrophilic phase chosen from water and mixtures of water and of hydrophilic organic solvent(s), such as alcohols and especially linear or branched lower monoalcohols containing from 2 to 5 carbon atoms, for instance ethanol, isopropanol, or n-propanol, and polyols, for instance glycerol, diglycerol, propylene glycol, sorbitol, pentylene glycol and polyethylene glycols.

44. Composition according to claim 43, in which the hydrophilic phase further contains hydrophilic C₂ ethers and C₂ to C₄ aldehydes.

45. Cosmetic composition according to claim 40, in which said physiologically acceptable medium further comprises a fatty phase composed of fatty substances that are liquid or solid at room temperature, of animal, plant, mineral or synthetic origin.

46. Composition according to claim 40, further comprising one or more cosmetically acceptable organic solvents.

47. Cosmetic composition according to claim 40, in which said physiologically acceptable medium further comprises one or more auxiliary film-forming agents chosen from plasticizers and coalescers.

48. Cosmetic composition according to claim 40, further comprising one or more dyestuffs chosen from water-soluble dyes and pulverulent dyestuffs, for instance pigments, nacres and flakes.

49. Composition according to claim 40, further comprising fillers.

50. Cosmetic composition according to claim 40, further comprising one or more ingredients commonly used in cosmetics, such as vitamins, thickeners, trace elements, softeners, sequestering agents, fragrances, acidifying or basifying agents, preserving agents, sunscreens, surfactants, antioxidants, agents for preventing hair loss, antidandruff agents and propellants, or mixtures thereof.

51. Cosmetic composition according to claim 40, characterized in that it is in the form of a suspension, a dispersion, a solution, a gel, an emulsion, especially an oil-in-water (O/W) or water-in-oil (W/O) emulsion, or a multiple emulsion (W/O/W or polyol/O/W or O/W/O emulsion), in the form of a cream, a paste, a foam, a dispersion of vesicles, especially of ionic or nonionic lipids, a two-phase or multi-phase lotion, a spray, a powder or a paste, especially a soft paste or an anhydrous paste.

52. Cosmetic composition according to claim 40, characterized in that it is a hair product, such as a lacquer or a shampoo.

53. Cosmetic composition according to claim 40, characterized in that it is a makeup composition, such as a nail varnish.

54. Cosmetic process for making up or caring for keratin materials, comprising the application to the keratin materials of a composition according to claim 40.

55. Use of the copolymer according to claim 1, to improve the styling power and the hold of a hair lacquer.

56. Use of the copolymer according to claim 1, to increase the adhesion and the wear resistance of a nail varnish.

57. Use of the copolymer according to claim 1, to improve the hold of a makeup composition.
